(19) United States
(12) Patent Application Publication
(10) Pub. No.: US 2026/0026907 A1
(43) Pub. Date: Jan. 29, 2026

(54) DEVICES FOR CONTROLLING AN ENDOVASCULAR SYSTEM

(71) Applicant: UAB TELEMEDICINA, Kaunas (LT)

(72) Inventors: Evaldas KALVAITIS, Kaunas (LT); Vilius DAMBRAUSKAS, Kaunas (LT); Vaidas LABUNSKAS, Kaunas (LT)

(73) Assignee: UAB TELEMEDICINA, Kaunas (LT)

(21) Appl. No.: 19/145,462

(22) PCT Filed: Jan. 15, 2024
(86) PCT No.: PCT/EP2024/050748

§ 371 Date: Jul. 2, 2025

(30) Foreign Application Priority Data
Feb. 28, 2023 (DE) 10 2023 104 921.6

(51) Int. Cl. A61B 34/00; A61B 34/35; A61B 90/00

(52) U.S. Cl. CPC A61B 34/76; A61B 34/25; A61B 34/35; A61B 34/77; A61B 90/06; A61B 2034/258; A61B 2090/064

(57) ABSTRACT

A human control unit for manipulating an apparatus for an endovascular procedure, the human control unit comprising: a housing; a control unit comprising a transceiver; a moveable member couplable to the housing and moveable by a user of the human control unit; and a haptic feedback unit communicatively couplable to the control unit and couplable to the moveable member; wherein, upon a movement of the moveable member, the transceiver is configured to provide a signal to the apparatus for altering a parameter of the apparatus, and wherein the haptic feedback unit is configured to provide haptic feedback via the moveable member to the user of the human control unit.

### FIELD OF THE INVENTION

The present invention generally relates to an apparatus for an endovascular procedure comprising a moveable base, a syringe and a control unit. Furthermore, the present invention generally relates to systems for an endovascular procedure and for manipulating an apparatus for an endovascular procedure and a human control unit (meaning a control unit controllable by a human) for manipulating an apparatus for an endovascular procedure.

### BACKGROUND TO THE INVENTION

Endovascular specialists (for example (endo-)vascular surgeons, (interventional) cardiologists, (interventional) radiologists etc.) train, practice and develop intuitive skills to handle surgical tools. The mental imagery of skills of physicians also evolves by correlating their actions and responses of surgical tools within the human anatomy. An endovascular surgeon is generally guided by two senses: visual feedback from the imaging devices and reaction force feedback via the tool. Perception-action-visualization abilities of surgeons are fine-tuned to a level where their surgical decisions are made even without observing their hand gestures.

Currently, existing robotic systems are focused exclusively on imaging feedback, but have ignored the other source of information: tactile feedback from surgical tools. Member controls using a joystick and a PC interface are closer to videogame controllers than control of surgical members and leave vascular surgeons with less feedback information which is available performing the procedure manually.

In particular, an indeflator is connected with an endovascular balloon catheter via tubing. The tubing, balloon catheter and indeflator are filled with fluid, via a fluid line. The pressure within the fluid line is increased by moving the indeflator plunger linearly forward, while linear motion is created by the user by turning the indeflator handle. As fluid is generally incompressible, each plunger movement in the linear direction significantly increases the pressure in the fluid line, and balloon catheter. For this reason, a rotational movement of the handle is used for reaching and keeping the wanted pressure in a precise manner. Precise pressure is needed to deploy, e.g., balloons/stents.

The indeflator also has a quick release button—by pressing it, the plunger can be pulled back by pulling the rotating hand backward manually and keeping the release button pressed. With this, the user can create a vacuum in the fluid line by pulling the handle back. This is used for safety reasons to ensure all air bubbles come to the distal part of the indeflator, or after the procedure has been completed and the balloon needs to be deflated and withdrawn from the patient.

The inventors have realized the need for improving, for example, manual indeflators, or generally (endo-)vascular systems.

There is therefore a need for improvements of (endo-)vascular systems, in particular, indeflators.

### SUMMARY OF THE INVENTION

The inventors have realized that existing (endo-)vascular systems, in particular manual indeflators, can be improved, for example, by adding certain remote control functionality in particular together with a haptic user interface.

The invention is set out in the independent claims. Preferred embodiments of the invention are set out in the dependent claims

According to a first aspect, we describe an apparatus for an endovascular procedure, wherein the apparatus comprises: a moveable base; a drive unit coupled to at least a first portion of the moveable base and configured to move the first portion of the moveable base; a syringe comprising a plunger, wherein the syringe is couplable to the base; and a first control unit configured to control a movement of at least a first portion of the movable base; wherein, upon an instruction provided by the first control unit to the drive unit, at least the first portion of the moveable base is configured to move the plunger from a first position to a second position and/or the second position to the first position, and wherein the first and second positions are different positions.

Throughout the present disclosure, the term “first position to second position”, or variants thereof, is used. It should be understood that when this term is used, the term “second position to first position”, or variants thereof, also apply. The same applies to any other mentioning of positions within the present disclosure.

Additionally, throughout the present disclosure, it is to be understood that the “proximal end” of the syringe is the end at which the elongated medical member is couplable to the syringe, and the “distal end” is the end of the syringe comprising the plunger.

The moveable base may allow for the syringe to be moved, thereby improving the efficiency and accuracy of the endovascular procedure. The base may also be moveable to accommodate different syringes, be it in diameter and/or length, so that the base is compatible with off-the-shelf syringes that can be commercially bought.

The drive unit may allow for at least a first portion of the moveable base to be moved, as will be described in more detail below. In some examples, the drive unit is integral to the base. In some examples, the drive unit is external to the base.

The elongated medical member may be a known elongated medical member such as a catheter, a catheter balloon, a stent balloon, or a thrombectomy device. The elongated medical member may be altered based on the endovascular procedure being undertaken by the apparatus.

The control unit may be configured to instruct various parts of the moveable base to move. This may allow for the efficiency and accuracy of the endovascular procedure to be increased, as a surgeon, or user, can complete the procedure from a remote location, as will be described in further detail below, and allow for a reduction in human error, as the apparatus is less likely to make unwanted moves and inputs during a procedure when compared to a human. In some examples, the control unit is integral to the base. In some examples, the control unit is external to the base.

The plunger may be moved from the first position to the second position, and vice versa, based on a movement of the first portion of the moveable base.

In some examples, the apparatus further comprises an elongated medical member couplable to a proximal end of the syringe. Preferably, the proximal end of the syringe comprises a standard luer lock adapter, in particular, a male lock. The elongated medical member may then have a corresponding female lock to couple the two elements to one another. The elongated medical member can therefore be manually disconnected from the proximal end of the syringe.

In some examples, the moveable base further comprises a connector configured to couple at least the plunger of the syringe to the first portion of the moveable base. This may allow for the syringe to be coupled to the moveable base. The connector may be of such a shape that allows for the plunger of the syringe to be coupled to the base and/or for the distal end of the syringe to be coupled to the base. In the case of the plunger being coupled to the base, the connector may be substantially C-shaped in order to allow for the column of the plunger to be inserted through the gap, and then the connector secures the plunger. Alternatively, the connector may be substantially U-shaped, substantially V-shaped, or comprise any suitable geometry that allows for the plunger of the syringe to be coupled to the base and/or for the distal end of the syringe to be coupled to the base. In some examples, the connector comprises a clamp configured to securely couple the plunger of the syringe to the base. Additionally, the main body of the syringe may be coupled to the base via a second connector, which may be a clamp, or other suitable means. The above connector may allow for the syringe to be kept in place during the procedure, thereby improving the safety of the procedure. In some examples, the connector may be a part of the first portion of the moveable base. In some examples, the connector may be the first portion of the moveable base.

In some examples, the drive unit comprises a linear gear coupled to the connector, and wherein the linear gear is configured to move the connector from a third position to a fourth position and/or the fourth position to the third position, upon the instruction being provided by the first control unit to the drive unit, wherein, when the connector is in the third position, the plunger is in the first position, and wherein, when the connector is in the fourth position, the plunger is in the second position. As the connector is coupled to the base and the plunger of the syringe, as the connector is moved, the plunger of the syringe is also moved. This then allows for liquid which may be within the syringe to be injected into a patient and/or for fluid to be extracted from a patient. The linear gear may be any suitable known linear gear that allows for precise, continuous linear movement of the connector. In some examples, this linear movement may be in a non-stepped manner. As the linear gear is able to be moved in very small increments, the connector is therefore moved in very small increments, thereby allowing for the syringe, and the apparatus, to be accurately and precisely used.

In some examples, the connector is configured to be moved from the third position to the fourth position and/or the fourth position to the third position, via the linear gear, when the instruction to move the connector from the third position to the fourth position and/or the fourth position to the third position is executed by the first control unit. The control unit may instruct the linear gear to actuate.

In some examples, the control unit comprises a processor and a memory, wherein the memory is configured to store an instruction and the processor is configured to execute the instruction stored in the memory and/or an instruction received from an external source. This may allow for the apparatus to be controlled in a predetermined manner, should the instruction be stored in the memory and/or remotely, should the instruction be received form the external source. The processor, and the control unit, may then instruct various parts of the apparatus to move in line with the executed instruction. This may then allow for the control unit, and the apparatus, to be controlled from a remote location, should the instruction be received from the external source.

In some examples, the drive unit comprises a rotational gear, and wherein the rotational gear is configured to rotate at least a second portion of the moveable base about a fixed point on and/or a fixed axis through the moveable base. The axis may be, for example, an axis of the syringe, an axis of the moveable base, an axis of the connection between the elongated medical member and the syringe, or any other suitable axis. In some examples, the second portion may be able to be rotated about a plurality of axes. The point may be, for example, a point on the syringe, a point on the moveable base, a point of connection between the elongated medical member and the syringe, or any other suitable point. The rotational gear may rotate the entire base, or, in some examples, a portion of the moveable base. Preferably, the portion of the moveable base that is rotated comprises the connector described above. The rotational gear may be any suitable known rotational gear that allows for continuous, controlled movement of the connector and/or the base. In some examples, this movement may be in a non-stepped manner. As the rotational gear is able to be moved in very small increments, the connector and/or the base is therefore moved in very small increments, thereby allowing for the syringe and the apparatus to be accurately and precisely used. In some examples, the first and second portions of the moveable base are different portions. In some examples, the first and second portions are the same portion of the moveable base. In some examples, one of the portions is encompassed by the other portion. That is to say, the first portion may be a sub-portion of the second portion, or vice versa.

In some examples, the rotational movement of the second portion of the moveable base is further configured to rotate the moveable base about a proximal end of the syringe. That is to say, the end of the syringe coupled to the elongated medical member is substantially kept in the same place during the rotation of the moveable base. This may therefore allow for the relative position of the elongated medical member with respect to both the syringe, a surface on which the apparatus is placed, and the patient to remain substantially stable. This may be important during procedures, as movement of the elongated medical member within the patient may cause unwanted effects.

In some examples, the second portion of the moveable base is configured to be rotated, via the rotational gear, when an instruction to rotate at least the second portion of the moveable base is executed by the first control unit. This may allow for the rotational gear to be actuated.

In some examples, at least the second portion of the movable base is configured to be repeatedly rotated, via the rotational gear, from a fifth position to a sixth position, and back to the fifth position, when an instruction to repeatedly rotate at least the second portion of the moveable base is executed by the first control unit. That is to say, upon execution of a single instruction, the moveable base may be repeatedly rotated through a predetermined angle. This may allow for any air bubbles within the syringe and/or the elongated medical member to be moved to within the syringe. This may be important as air bubbles within the elongated medical member and/or syringe may cause unwanted effects to the patient during the procedure. Resultantly, the procedure may be safer.

In some examples, the moveable base further comprises an actuatable device, wherein, upon actuation of the actuatable device, the first control unit is configured to execute an instruction to move the connector from the third position to the fourth position and/or the fourth position to the third position and/or rotate at least the second portion of the moveable base. This is to say, there may be an actuatable device on the base that may be actuated by a user. Upon actuation of the device, the base may be rotated and/or the connector may be moved from the third position to the fourth position. This may be particularly helpful when coupling a new syringe to the base, or when removing a syringe from the base. In some examples, there are multiple actuatable devices. There may be a first device for moving the connector toward the distal end of the syringe and/or a second device for moving the connector towards the proximal end of the syringe and/or a third device for rotating at least a portion of the base in a first direction and/or a fourth device for rotating at least a portion of the base in a second direction and/or a fifth device to reposition at least the portion of the base and/or the connector to a predetermined position. This may allow for any commercially available syringe to be used in combination with the apparatus described herein.

In some examples, the apparatus further comprises a safety sensor couplable to the control unit, and wherein the safety sensor is configured to determine if the syringe is coupled to the moveable base. This safety sensor may work in a similar manner to a dead man's switch. This is to say, the sensor may detect when the syringe is coupled to the moveable base, and allow for the base/apparatus to be used when the syringe is detected to be in place. Conversely, when the safety sensor detects that the syringe is not coupled to the base, it may disable the base. The safety sensor may be any one or more of a Hall sensor, a distance sensor, a light sensor, a depressible button, and any other suitable method for allowing the determination of the coupling between the syringe and the base.

In some examples, the apparatus further comprises a first pressure sensor communicatively couplable to the first control unit, wherein the first pressure sensor is couplable to the syringe, and wherein the first pressure sensor is configured to measure a pressure within the syringe based on a movement of the plunger of the syringe, wherein the first pressure sensor is configured to measure pressure difference between the pressure within the syringe and a predetermined pressure value. This may allow for the pressure being exerted within the syringe and/or by a fluid which may be within the syringe, to be calculated. For example, the control unit may know the area of the syringe, and know the force currently being used to move the connector to its present position when compared to its initial position. The force may be calculated by a force cell located on the connector and/or on the base. In this case, the cross-sectional area of the syringe is known and so, the first pressure sensor can measure the pressure via the equation P=F/A, wherein P is the pressure, F is the force measured by the force cell, and A is the cross-sectional area of the syringe. The control unit may then be able to calculate the pressure being exerted. This pressure sensor may be within the base, or external to the base, and couplable to the control unit via wired and/or wireless means. This may then give the user an accurate indication of the pressure currently being exerted, should the user not be located within the same room as the room where the procedure is taking place. The pressure difference may be determined from a predetermined point such as, for example, 1 atmosphere, 1 bar, or any other suitable starting point.

In some examples, the first pressure sensor is configured to measure the pressure based on a movement of the connector from the third position to the fourth position and/or the fourth position to the third position. The third position may be an initial “zero” position where the pressure is 1 bar or 1 atmosphere.

In some examples, the syringe is at least partially filled with a liquid. This may be useful for procedures where a dye may need to be used, where a catheter balloon may need to be used, or for IV injection of a fluid. In some examples, saline or other fluids, such as liquids and/or gases, can be placed in the syringe. As a non-limiting example, if the apparatus is used in conjunction with a closed balloon catheter, contrast media can be used via the open catheter and microspheres can simultaneously be used. Alternatively, by creating vacuum, the syringe can act as a suction pump. This, therefore, may allow for the apparatus to be useable in a large range of endovascular procedures.

In some examples, the liquid comprises microspheres. The skilled person understands that microspheres are particularly sized particles that, when in liquid, are used for the closure of small vessels after being delivered from the syringe, via an open catheter, to the particular place in a patient. Microspheres are preferably used in conjunction with the repeated rotation of the base mentioned herein, as this may allow for the microspheres to be evenly distributed thorough the liquid, and for the microspheres to be precisely delivered. Additionally or alternatively, glue and/or a coil may be used as these also result in the closure of vessels. Each of these options can be considered to be embolization materials/instruments.

In some examples, the apparatus further comprises a second pressure sensor communicatively couplable to the first control unit, wherein the second pressure sensor is couplable to the elongated medical member, wherein the elongated medical member is at least partially filled with the liquid, and wherein the second pressure sensor is configured to measure a pressure of the liquid within the elongated medical member. This pressure sensor may work in a similar way to the first pressure sensor mentioned above, but in this case, the pressure sensor directly measures the pressure of the fluid/liquid in the elongated medical member, and does not use the force or area to calculate this pressure. This pressure reading may be used as a safety reading, should the first pressure sensor fail, or be used to compare the readings. If the readings are too far apart, an audio and/or haptic and/or visual indication may be given to a user of the apparatus to indicate this. Additionally or alternatively, the control unit of the apparatus may stop the movement of the linear and/or rotational gear until the discrepancy has been rectified. The second pressure sensor may be couplable to the control unit via wired and/or wireless means. This therefore improves the safety of the apparatus.

In some examples, the apparatus further comprises a third pressure sensor configured to measure a force exerted by the plunger on the syringe. This may be used in combination, or separately, to the first and/or second pressure sensor mentioned above. This third pressure sensor may measure the force, and indicate this to the user and/or be used in the pressure calculation of the first pressure sensor. If this is a separate element, the control unit may compare the estimated reading of the force from the movement of the connector, and the reading of the third pressure sensor. If the readings are too far apart, an audio and/or haptic and/or visual indication may be given to a user of the apparatus to indicate this. Additionally or alternatively, the control unit of the apparatus may stop the movement of the linear and/or rotational gear until the discrepancy has been rectified. The third pressure sensor may have a similar design to the first pressure sensor mentioned above, but be placed on the outer casing of the syringe. The third pressure sensor may therefore be used for safety purposes, and as a duplication of the first pressure sensor. That is to say, the first pressure sensor may measure the force needed to push the plunger, and the second pressure sensor may measure the force acting on the casing of the syringe. This therefore improves the safety of the apparatus.

In some examples, a distal end of the syringe is configured to be elevated above the proximal end of the syringe during use of the apparatus, wherein the proximal end of the syringe is an end at which the elongated medical member is couplable to the syringe, and the distal end is an end of the syringe comprising the plunger, wherein the distal and proximal ends are at two different end portions of the syringe. This, in turn, improves the safety of the procedure, as air bubbles may cause unwanted effects to the patient during the procedure. In some examples, the distal end of the syringe could be above the proximal end at all times during use of the apparatus.

In some examples, the apparatus further comprises an expandable medical member couplable to the elongated medical member, wherein a first end of the elongated medical member is couplable to the proximal end of the syringe, and the expandable medical member is couplable to a second end of the elongated medical member, wherein the first and second ends are at opposite ends of the elongated medical member. That is to say, the expandable medical member may be a balloon, or any other suitable expandable member. This may be particularly helpful during angioplasty procedures.

In some examples, a diameter of the expandable medical member is dependent on a pressure of a liquid within the elongated medical member. Therefore, the pressure readings from the pressure sensors mentioned above may be helpful for the user determining what the diameter of the expandable medical member is, thereby improving the safety of such a procedure.

In some examples, the apparatus is additionally or alternatively suitable for angioplasty. In particular, the apparatus may be used in coronary angioplasty and/or peripheral angioplasty and/or carotid angioplasty.

In some examples, the apparatus is additionally or alternatively suitable for embolization. If used in conjunction with the expandable medical member mentioned above, this may allow for tumors and/or aneurysms to be treated, thereby improving the wellbeing of the patient. The skilled person understands that different sizes of syringe are used for embolization. Therefore, via the apparatus being suitable for any commercially available syringe, the apparatus can also be used for embolization. In some examples, if the apparatus is suitable for embolization, this may be used in conjunction with the rotation of the base described herein. Additionally or alternatively, the user interface (described in more detail below) may indicate volume rather than pressure.

According to a second aspect, we describe a system for endovascular procedures comprising: the apparatus according to any one of the examples as described above; and a human control unit comprising a second control unit; wherein the human control unit is located at a first location and the apparatus is located at a second location; and wherein the first and second locations are different locations.

The human control unit may be located at a location remote from the apparatus and be coupled to the apparatus via wired and/or wireless means. This may allow for the apparatus to be controlled remotely. This may allow for improved reaction times to critical procedures, should a qualified surgeon not be present at the location of the apparatus, a reduction in the use of surgeon PPE, and a reduction in fatigue of the surgeon due to reduced travel times between their place of work or living, and the place of the procedure.

According to a third aspect, we describe a human control unit for manipulating an apparatus for an endovascular procedure, the human control unit comprising: a housing; a control unit comprising a transceiver; a moveable member couplable to the housing and moveable by a user of the human control unit; and a haptic feedback unit communicatively couplable to the control unit and couplable to the moveable member; wherein, upon a movement of the moveable member, the transceiver is configured to provide a signal to the apparatus for altering a parameter of the apparatus, and wherein the haptic feedback unit is configured to provide haptic feedback via the moveable member to the user of the human control unit.

The housing may be any suitable shape or design that allows for the human control unit to comprise the features described herein.

The control unit may be configured to instruct various parts of the base to alter states. In some examples, the control unit instructs an external apparatus to alter states. This may allow for the efficiency and accuracy of the endovascular procedure to be increased, as a surgeon, or user, can complete the procedure from a remote location, as will be described in further detail below, and allow for a reduction in human error, as the apparatus is less likely to make unwanted moves and inputs during a procedure when compared to a human.

The moveable member may be of any suitable design, as described below, that allows for said member to be moved. In particular, the moveable member can be a rotatable member, a slidable member, or any other suitable moveable member.

Throughout the present disclosure, a rotatable member is described, but it should be understood that the same effects and features can be applied to, for example, a slidable member. Therefore, whenever a rotatable member is described in the present disclosure, this equally applies to a slidable member or any other suitable moveable member.

In some examples, the haptic feedback provided to the user changes based on the altered parameter. This may allow for the user of the human control unit to feel when the parameter has been altered. In turn, this may improve the manipulation of the apparatus as the user can accurately feel what is happening at the apparatus.

In some examples, the moveable member comprises a rotatable member, and wherein the movement of the moveable member comprises a rotation of the rotatable member.

In some examples, the human control unit comprises a first actuatable device, wherein upon actuation of the actuatable device, the human control unit changes from a first mode to a second mode. In some examples, it may change from a first mode that relates to angioplasty, to a second mode that relates to embolization. In some examples, the first and second modes may relate to a standby mode and an active mode, a testing mode and an operating mode, or any other two suitable modes. This may allow for the human control unit to be used for a wide range of operations and procedures.

In some examples, the human control unit is couplable to the apparatus. This may allow for a user of the human control unit to remotely control the apparatus.

In some examples, the transceiver is further configured to receive an apparatus signal from the apparatus. This may allow for the human control unit to receive a controlling signal or receive a status indication from the apparatus.

In some examples, upon a movement of the moveable member, a parameter of the apparatus is altered. This may allow for the user to accurately and precisely control the parameter.

In some examples, the moveable member comprises a plurality of predefined positions, wherein the moveable member is clickable between said plurality of predefined positions and/or wherein the moveable member is continuously moveable. The moveable member may be “clickable” between the preset positions. That is to say, the moveable member may snap from one preset position to another upon movement (e.g. rotation). This “clickable” movement may be achieved by the moveable member being set on a pin, which is connected to a stepper motor. Resultantly, for each click of the moveable member, the stepper motor is moved by one step. Additionally or alternatively, the member may be moveable continuously and comprise a, theoretically, infinite number of positions. In some examples, the first and second modes mentioned above could relate to the preset positions in the first mode and the continuous rotation in the second mode. In some examples, there may be a mixture of predefined positions and continuous movement. For example, for a first portion of the movement, predefined positions may be used as the alteration to the parameter due to said movement is not important. Then, for a second position of the movement, the alterations to the parameter may need to be finely tunable and so, continuous movement is used. The above examples may lead to an improvement in safety of the procedure, as the parameter may not be allowed to exceed a predetermined limit.

In some examples, if the moveable member is “clickable”, the user may be able to predetermine the number of clicks needed to reach a predetermined pressure value. That is to say, the user can set the upper pressure value limit, and when this limit is reached, the maximum possible resistive force created by the stepper motor can be exerted to the moveable member, meaning the user cannot move the moveable member any further. This may improve the safety of the manipulation as the parameter cannot be altered beyond a limit.

In some examples, the moveable member is configured to provide haptic feedback to the user of the human control unit. This may allow for the user to have a realistic feel of the forces that are occurring at the apparatus. This haptic feedback is preferably proportional to a pressure value measured at the apparatus. In particular, this pressure may be the pressure measured by the first and/or second and/or third pressure sensors mentioned above. This may mean that for each increase in pressure, the resistive force felt by the user is increased. This may increase the accuracy of the procedure, as the user can feel what is happening at the apparatus. Although here, and throughout the present application, pressure is mentioned, it is to be understood that any other parameter, such as, for example, distance and/or force, may result in the same effects.

In some examples, the haptic feedback provided to the user changes based on the altered parameter. This is to say, as the parameter is altered, the haptic feedback is also altered. This may increase the accuracy of the procedure as the user can feel what is happening at the apparatus.

In some examples, upon the parameter increasing, the haptic feedback unit is configured to increase a force applied via the moveable member to the user. As a nonlimiting example, should the parameter be 0 N, the user may not feel any haptic feedback. As the parameter increases to 5 N, the user may feel a haptic force of 2 N acting on the moveable member, and as the parameter increases to 10 N, the force felt by the user may be 4 N. This may again help the user control the procedure, as at high (above a threshold) parameters, it may be difficult to rotate the member. In some examples, the force increase may be step-wise, or it may be a continuous curve. This, in turn, may improve the safety of the procedure, as the user may not be able to alter the parameter to dangerous levels.

In some examples, a change in the haptic feedback provided to the user is non-linear with respect to a change of a value of the parameter. As a non-limiting example, should the parameter be 0 N, the user may not feel any haptic feedback. As the parameter increases to 5 N, the user may feel a haptic force of 2 N acting on the moveable member, and as the parameter increases to 10 N, the force felt by the user may be 6 N. In some examples, the force increase may be step-wise, or it may be a continuous curve. This may again help the user control the procedure, as at high parameters, it may be difficult to rotate the member. This, in turn, may improve the safety of the procedure as the user may not be able to alter the parameter to dangerous levels.

The non-linear behavior with respect to the haptic feedback outlined above may, in some examples, comprise that an increase in force applied by the surgeon by a factor x result in an increase in force seen at the patient side by a factor of y, with x being larger than y. This may allow for avoiding unwanted effects based on too large pressures on the patent side, in particular when the surgeon operates from a remote location.

In some examples, a scalability of the moveable member changes as the parameter changes. As an example, if the parameter relates to force, and the member is rotatable, in order to go from 0 N to 5 N, a single full rotation of the rotatable member may be needed. Between 5 N and 10 N, 2 full rotations of the rotatable member may be needed, i.e. each rotation increases the force by 2.5 N. After 10 N, a single full rotation of the rotatable member may be needed in order to increase the force by 1 N. In some examples, the scalability may be step-wise, or it may be a continuous curve. This, in turn, may improve the safety of the procedure as the user may not be able to alter the parameter to dangerous levels.

In some examples, the human control unit further comprises a display couplable to the control unit and the housing. The display may be a known display apparatus, such as a tablet computer couplable to the housing, or an integrated display. Additionally or alternatively, the display may be a hologram and/or comprise an augmented reality element.

In some examples, the display is configured to display the parameter or a value associated with the parameter of the apparatus. This may allow for a user of the human control unit to see, and control, a wanted parameter. The parameter, both in this example and throughout the present disclosure, may be any suitable parameter such as, for example, distance, force, pressure, volume remaining in a syringe or line, a syringe plunger position, a syringe diameter, a detection of air bubbles in a syringe or line, a device mode/state and an alarm message and the like, or any combination thereof. In particular, the parameter may be a volume of a liquid remaining in the syringe and/or the elongated medical member. This may improve safety as it allows for the user of the human control unit to see that there is no/little liquid left, and therefore refrain from depressing the plunger of the syringe further (which may potentially result in a gas, such as air, being inserted into the patient, which is to be avoided).

In some examples, the human control unit further comprises a timing module configured to be activated upon an actuation of a second actuatable device, and wherein the display is configured to display an output of the timing module. This may allow the user to control time-sensitive operations and see how long a current procedure has been going on for. In some examples, the second actuatable device is integral to the human control unit. In some examples, the second actuatable device is external to the human control unit.

In some examples, the display is further configured to display a graph with the output of the timing module on a first axis, and (a value of) the parameter of the apparatus on a second axis. This may allow the user to control time- and parameter-sensitive operations and see how long a current procedure has been going on for, and how long the presently controlled parameter has been at its current level. In some examples, the first and second mode mentioned above may relate to switching between a first visualization and a second visualization, or a first parameter to be controlled and a second parameter to be controlled.

In some examples, the human control unit is configured to control a characteristic of the apparatus upon actuation of a third actuatable device. For example, the characteristic may relate to a movement of the apparatus, an activation of a feature, a deactivation of a feature, or any other suitable characteristic.

In some examples, the moveable member is single use and/or disposable. This may allow for sterility to be maintained. In particular, the moveable member may comprise any suitable plastic and/or metal.

In some examples, the moveable member comprises a circular cross-section or a cross shaped cross-section. This may be particularly advantageous, as the skilled person can alter the parameter more finely, thereby improving the accuracy of the manipulation of the apparatus, and the safety of the procedure. Additionally, this may help the moveable member to be rotated quicker, if needed, and also help to move the movable member should the user be wearing gloves. Additionally, the alterability of the moveable member, and therefore the cross-section, may allow for users to switch moveable members depending on personal preference. Although circular and cross cross-sections are mentioned, the skilled person understands that the cross-section can be any suitable cross-section. In some examples, the moveable member may further comprise dimples to help rotation, and to orientate the user.

In some examples, the human control unit further comprises a light source, and wherein at least one characteristic of the light source changes based on a change of a value of the parameter of the apparatus. This light source may give a visual indication to the user as to the state of the parameter. For example, if the parameter is within normal limits, a green light may be indicated. As the parameter gets close to its upper allowable limit, the light may turn yellow, and as the parameter exceeds its limit, the light may turn red. This may allow for the user to refrain from exceeding the limits of a parameter. The light source may be configured to indicate to the user of the human control unit an operating state of the apparatus. The operating state may relate to a critical pressure within the apparatus, or a single component in, or coupled to, the apparatus. Additionally or alternatively, additional haptic feedback may be provided by vibrating the moveable member, similar to a stick-shaker in an aircraft, and/or an audio indication may also be given. This may improve the safety of the procedure, as the user is given warning of when limits are going to be exceeded/have been exceeded.

According to a fourth aspect, we describe a system for manipulating an apparatus for endovascular procedures, the system comprising: an apparatus; and the human control unit according to any one of the example implementations as described above; wherein the human control unit is located at a first location and the apparatus is located at a second location; and wherein the first and second locations are different locations.

The human control unit may be located at a location remote from the apparatus and be coupled to the apparatus via wired and/or wireless means. This may allow for the apparatus to be controlled remotely. This may allow for improved reaction times to critical procedures, should a qualified surgeon not be present at the location of the apparatus, a reduction in the use of surgeon PPE, and a reduction in fatigue of the surgeon due to reduced travel times between their place of work or living, and the place of the procedure.

That is to say, the apparatus of the first aspect may be controlled by the human control unit of the third aspect, and that the apparatus and the human control unit, as described herein, may function as a system.

In some examples, the apparatus and the human control unit are communicatively couplable to one another wirelessly. This may allow for the apparatus and the human control unit to be located in two separate locations at a great distance from one another.

In some examples, the apparatus comprises a second transceiver, wherein the parameter alterable by the movement of the moveable member is a pressure, and wherein upon the pressure falling below a predetermined pressure, the apparatus is configured to send a signal to the human control unit for preventing the human control unit from manipulating the apparatus. This may be used to completely deflate the expandable medical member described above. This may also improve the safety of the procedure, as this ensures that the expandable medical member is fully deflated before being extracted from a patient.

In addition, or as an alternative, to any of the above-mentioned aspects, the current disclosure may relate to an indeflator, and the processes of using the indeflator, as mentioned in the background section herein. Any of the above aspects may relate to the same functionality and usage principle, but from a remote location. This may mean that the user can move the moveable member at a remote location, i.e. the human control unit, and the connector of the apparatus move slowly and precisely in a linear manner.

The syringe and apparatus may, in principle, be similar to an infusion pump. That is to say, a standard syringe may be inserted into an inflation module and connected, via a fluid line, to a balloon catheter. Injection of a fluid from the syringe during use of an inflation module needs to be very slow, and comprise constant and accurate dosing of the liquids. The disclosure described herein may be able to achieve this same function from a remote location, thereby allowing a user to administer fluids to many patients simultaneously while monitoring said patients.

Additionally, present standard manual indeflators require more force to turn the handle as the pressure increases. In the apparatus or system according to any of the above-mentioned aspects, the same haptic feedback may be provided to the moveable member. The member may be more difficult to move as the pressure in the fluid line increases.

Any advantages and features described in relation to the any of the above aspects and examples may be realized in any of the other aspects and examples described above.

It is clear to a person skilled in the art that certain features of the system set forth herein may be implemented under use of hardware (circuits), software means, or a combination thereof. The software means can be related to programmed microprocessors or a general computer, an ASIC (Application Specific Integrated Circuit) and/or DSPs (Digital Signal Processors). For example, a processing unit may be implemented at least partially as a computer, a logical circuit, an FPGA (Field Programmable Gate Array), a processor (for example, a microprocessor, microcontroller (uC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

Even if some of the aspects described above have been described in reference to any one of the first to fourth aspects, these aspects may also apply to a method (in particular of controlling an apparatus for endovascular procedures) and vice versa.

### BRIEF DESCRIPTION OF THE DRAWINGS

These and other aspects of the invention will now be further described, by way of example only, with reference to the accompanying figures, wherein like reference numerals refer to like parts, and in which:

FIG. 1 shows a schematic view of an apparatus for endovascular procedures according to some example implementations as described herein;

FIG. 2 shows a schematic view of an apparatus for endovascular procedures according to some example implementations as described herein;

FIG. 3 shows a schematic view of an apparatus for endovascular procedures according to some example implementations as described herein;

FIG. 4 shows a block diagram of an apparatus for endovascular procedures according to some example implementations as described herein; and

FIG. 5 shows a block diagram of a human control unit (surgeon unit) according to some example implementations as described herein.

### DETAILED DESCRIPTION OF THE PREFERRED EMBODIMENTS

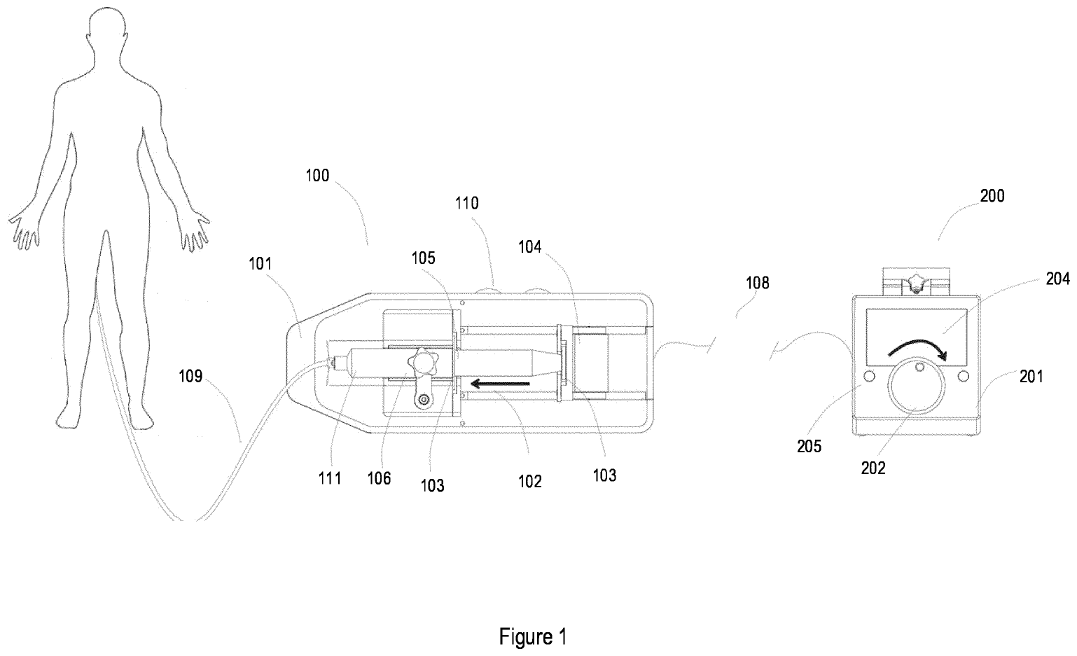

FIG. 1 shows a schematic view of an apparatus for endovascular procedures according to some example implementations as described herein.

FIG. 1 shows an apparatus 100 for endovascular procedures and a human control unit 200 for manipulating the apparatus 100 for endovascular procedures. The apparatus controlled by the human control unit 200 is, in this example, the apparatus 100 shown. In some examples, the human control unit 200 may control any suitable type of apparatus for endovascular procedures that can be remotely controlled.

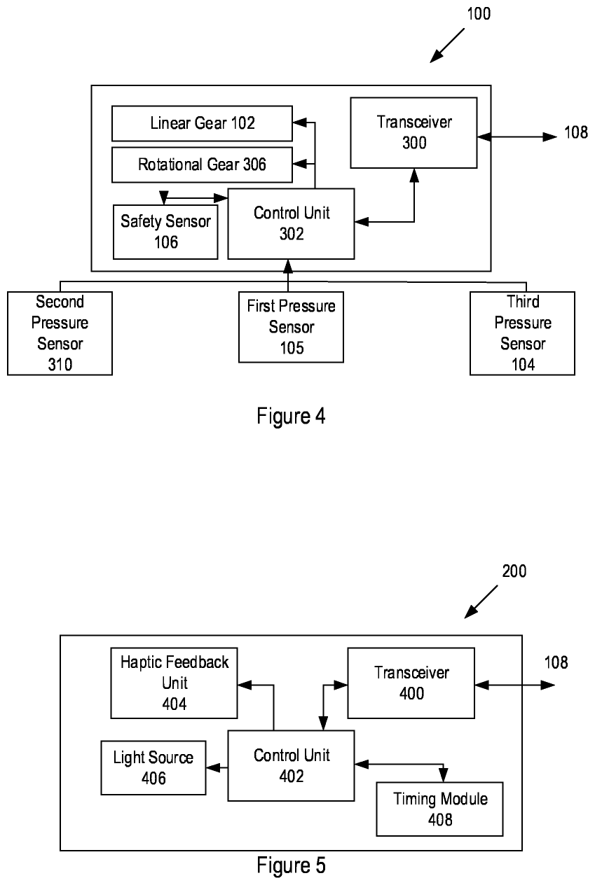

In this example, the apparatus 100 comprises a moveable base 101, a linear gear 102, a connector 103, a third pressure sensor 104, a first pressure sensor 105, a safety sensor 106, a control unit (shown in FIG. 4), a communication line 108, an elongated medical member 109, an actuatable device for controlling movement of at least the linear gear 110 and a syringe 111. In addition, the apparatus 100 comprises a rotational gear, a transceiver and a secondary pressure sensor (all shown in FIG. 4). The human control unit 200 comprises a housing 201, a rotatable member 202, a display 204 and an actuatable device 205. The human control unit 200 further comprises a transceiver, a control unit, a haptic feedback unit, a light source and a timing module (all shown in FIG. 5).

In the example of FIG. 1, the syringe 111 is couplable to the base 101 via a connector 103. The connector 103 may be of such a shape that allows for the plunger of the syringe 111 to be coupled to the base 101 and/or for the distal end of the plunger to be coupled to the base 101. In the case of the distal end of the plunger being coupled to the base 101, the connector 103 may be substantially C-shaped in order to allow for the column of the plunger to be inserted through the gap, and then the connector 103 secures the distal end of the plunger. Alternatively, the connector 103 may be substantially U-shaped, substantially V-shaped, or comprise any suitable geometry that allows for the plunger of the syringe 111 to be coupled to the base 101 and/or for the distal end of the syringe 111 to be coupled to the base 101. In some examples, the connector 103 comprises a clamp configured to securely couple the plunger of the syringe 111 to the base 101. Additionally, the main body of the syringe 111 may be coupled to the base 101 via a second connector, which may be a clamp, or other suitable means. The above connector 103 may allow for the syringe 111 to be kept in place during the procedure, thereby improving the safety of the procedure.

At the proximal end of the syringe 111, an elongated medical member 109 is couplable to said proximal end. The elongated medical member 109 may be a catheter, a catheter balloon, a stent balloon, a thrombectomy device, a glue system, according to the procedure the apparatus 100 is being used for. The syringe 111 and the elongated medical member 109 may be couplable by, preferably, the proximal end of the syringe 111 comprising a standard luer lock adapter, in particular, a male lock, and the elongated medical member 109 comprising a corresponding female lock to couple the two elements to one another.

The control unit of the apparatus 100 comprises a processor and a memory, wherein the memory is configured to store an instruction and the processor is configured to execute the instruction stored in the memory and/or an instruction received from an external source. In some examples, the external source is the human control unit 200 described herein. In some examples, the external source may be any other device suitable for controlling the apparatus 100 from a remote location such as, for example, a computer. The communication line 108 may couple the apparatus 100 to the external source and use any suitable wired and/or wireless method in order to communicate with the external source.

The base further comprises a linear gear 102. The linear gear may be any commercially available linear gear, or a bespoke linear gear. The linear gear may comprise a set of gears, hydraulic means, electrical means, or any other suitable means for linearly moving the connector 103. In some examples, the base 101 compresses an opening along a portion of the base 101 in which a first end of the connector 103 is couplable to the linear gear 102 inside the base 101 and in which a second end of the connector 103 is outside of the base 101 and is suitable for at least coupling the plunger of the syringe 111 to the connector. In this example, the opening allows for the connector 103 to linearly move from one end of the base 101 to the other, while depressing and/or releasing the plunger of the syringe 111.

Upon an instruction from the control unit, the linear gear 102 may actuate and move the connector 103 from a third position to a fourth position. This may allow for the connector 103 to move linearly and so, for the plunger of the syringe 111 to be released/depressed from the first position to the second position, or vice versa. Connections between gears and control units, and methods of causing gears to undergo instructions executed by the control unit, are known to the skilled person. In some examples, there may be a plurality of linear gears. This movement may be in the direction of the arrows shown in FIG. 1.

In this example, the base 101 also comprises an actuatable device 110. This actuatable device 110, upon actuation by a user, may allow for the connector 103 to linearly move and/or for the rotational gear to be actuated, the result of which is described in more detail below. This may allow for the connector 103 and/or the rotational gear to be actuated manually. This may be particularly helpful during loading and unloading of the syringe 111. Due to this, the apparatus 100 may be compatible with any off-the-shelf syringe 111, as the apparatus 100 can accommodate different sizes of syringe 111 due to the actuatable device 110. In some examples, there are multiple actuatable devices 110. There may be a first device 110 for moving the connector 103 toward the distal end of the syringe 111 and/or a second device 110 for moving the connector 103 towards the proximal end of the syringe 111 and/or a third device 110 for rotating at least a portion of the base 101 in a first direction and/or a fourth device 110 for rotating at least a portion of the base 101 in a second direction and/or a fifth device 110 to reposition at least the portion of the base 101 and/or the connector 103 to a predetermined position. This may allow for any commercially available syringe to be used in combination with the apparatus described herein.

The apparatus 100, in this example, also comprises a safety sensor 106. This safety sensor 106 may work in a similar manner to a dead man's switch. This is to say, the sensor 106 may detect when the syringe 111 is coupled to the moveable base 101, and allow for the base 101/apparatus 100 to be used when the syringe 111 is detected to be in place. Conversely, when the safety sensor 106 detects that the syringe 111 is not coupled to the base 101, it may disable the base 101. The safety sensor 106 may be any one or more of a Hall sensor, a distance sensor, a light sensor, a depressible button, and any other suitable method for allowing the determination of the coupling between the syringe 111 and the base 101.

In this example, the apparatus 100 further comprises a first pressure sensor 105. The first pressure sensor is preferably couplable to couplable to the control unit and the syringe 111, and configured to measure a pressure exerted by the syringe 111 based on a movement of the plunger of the syringe 111, wherein the pressure is measured from a predetermined pressure value. The predetermined value may be determined from a preset value stored within the memory of the control unit and/or on the basis of the pressure exerted when the safety sensor 106 indicates that the syringe 111 is securely coupled to the base 101 and/or after the connector 103 has moved a predetermined distance. This may allow for the pressure being exerted by the syringe 111 and/or a fluid which may be within the syringe, to be calculated. For example, the control unit may know the area of the syringe 111, and know the force currently being used to move the connector 103 to its present position when compared to its initial position. The force may be calculated by a force cell located on the connector 103 and/or on the base 101. In this case, the cross-sectional area of the syringe 111 is known and so, the first pressure sensor 105 can measure the pressure via the equation P=F/A, wherein P is the pressure, F is the force measured by the force cell, and A is the cross-sectional area of the syringe 111. The control unit may then be able to calculate the pressure being exerted. This pressure sensor 105 may be within the base 101, or external to the base 101, and couplable to the control unit wired and/or wirelessly. This may then give the user an accurate indication of the pressure currently being exerted, should the user not be located within the same room as the room where the procedure is taking place. The pressure may be calculated based on a movement of the connector 103 from the third position to the fourth position. The third position may be an initial “zero” position where the pressure is 0 bar, or 1 bar, or 1 atmosphere.

In this example, the apparatus 100 further comprises a third pressure sensor 104. The third pressure sensor 104 may be configured to measure a force exerted, by the plunger, on the syringe 111 and/or a fluid within the syringe 111. This may be used in combination, or separately, to the first pressure sensor 105 mentioned above. This third pressure sensor 104 may measure the force, and indicate this to the user and/or be used in the pressure calculation of the first pressure sensor 105. If the third pressure sensor 104 is a separate element, the control unit may compare the estimated reading of the force from the movement of the connector 103 via the linear gear 102, and the reading of the third pressure sensor 104. If the readings are too far apart, an audio and/or haptic and/or visual indication may be given to a user of the apparatus 100 to indicate this. Additionally or alternatively, the control unit of the apparatus 100 may stop the movement of the linear and/or rotational gear until the discrepancy has been rectified. This therefore improves the safety of the apparatus.

In some examples, the syringe is at least partially filled with a liquid. This may be useful for procedures where a dye may need to be used, where a catheter balloon may need to be used, or for IV injection of a fluid. In some examples, saline or other fluids, such as liquids and/or gases, can be placed in the syringe 111. As a non-limiting example, if the apparatus 100 is used in conjunction with a closed balloon catheter, contrast media can be used via the open catheter and microspheres can simultaneously be used. Alternatively, by creating vacuum, the syringe 111 can act as a suction pump. This, therefore, may allow for the apparatus to be useable in a large range of endovascular procedures. In some examples, the liquid comprises microspheres.

In some examples, the apparatus comprises a second pressure sensor. This second pressure sensor is preferably couplable to the control unit and the elongated medical member 109, and is configured to measure a pressure of the liquid within the elongated medical member 109. This pressure sensor may work in a similar way to the first pressure sensor 105 mentioned above, but in this case, the pressure sensor directly measures the pressure of the fluid/liquid in the elongated medical member 109, and does not use the force being provided by the plunger of the syringe 111 or the area of the syringe 111 to calculate this pressure. This pressure reading may be used as a safety reading, should the first pressure sensor 105 fail, or be used to compare the readings. If the readings are too far apart, an audio and/or haptic and/or visual indication may be given to a user of the apparatus 100 to indicate this. Additionally or alternatively, the control unit of the apparatus may stop the movement of the linear and/or rotational gear until the discrepancy has been rectified. This therefore improves the safety of the apparatus. In some examples, the second pressure sensor is an optional sensor to be used in conjunction with the first and/or third pressure sensors 104, 105. That is to say, the first and third pressure sensors 104, 105 compare readings for safety and stability reasons. The second pressure sensor acts as a direct pressure sensor in a fluid line coupled to the apparatus to measure the pressure in a fluid line.

During use of the apparatus 100, it is preferable for the distal end of the syringe 111 to be above the proximal end of the syringe 111. This may reduce the number of air bubbles that may enter the elongated medical element 109 and/or collate at the proximal end of the syringe 111. This, in turn, improves the safety of the procedure as air bubbles may cause unwanted effects to the patient during the procedure.

In some examples, the apparatus 100 further comprises an expandable medical member couplable to the elongated medical member 109, wherein a first end of the elongated medical member 109 is couplable to the proximal end of the syringe 111, and the expandable medical member is couplable to a second end of the elongated medical member 109, wherein the first and second ends are at opposite ends of the elongated medical member 109. That is to say, the expandable medical member may be a balloon, or any other suitable expandable member. This may be particularly helpful during angioplasty procedures. In some examples, a diameter of the expandable medical member is dependent based on the pressure of the liquid within the elongated medical member 109. Therefore, the pressure readings from the pressure sensors 105 mentioned above may be helpful for the user determining what the diameter of the expandable medical member is, thereby improving the safety of such a procedure. Many commercially available balloons are provided with a chart that correlates pressure with the diameter of the balloon. This, in turn, allows for a user of the apparatus to accurately determine the diameter of the balloon and so, successfully provide the required treatment to the patient. The user may be able to view the pressure, in terms of bar or atmospheres, and then be able to determine the diameter of the balloon. In some examples, there is a display on the apparatus 100 indicating the present pressure. In some examples, the pressure is displayed on the display 204 of the human control unit 200.

The human control unit 200, in this example, comprises a housing 201, a rotatable member 202, a display 204 and an actuatable device 205. The human control unit 200 further comprises a transceiver, a control unit, a haptic feedback unit, a light source and a timing module (all shown in FIG. 5). The human control unit 200 also comprises a communication line 108. The communication line 108 may couple the apparatus 100 to the human control unit 200 and use any suitable wired and/or wireless method in order to communicate with the apparatus 100. In some examples, the communication line 108 is not used to communicate with the apparatus 100, but with any suitable device that can be remotely controlled.

Upon actuation of the actuatable device 205, the human control unit 200 may change from a first mode to a second mode. The first mode may relate to angioplasty and the second mode may relate to embolization. In some examples, the first and second modes may relate to a standby mode and an active mode, a testing mode and an operating mode, a preset position rotation of the rotatable member 202 in the first mode and a continuous rotation of the rotatable member 202 in the second mode, switching between a first visualization and a second visualization on the display 204, a first parameter to be controlled and a second parameter to be controlled or any other two suitable modes. This may allow for the human control unit 200 to be used for a wide range of operations and procedures. In some examples, there may be a plurality of actuatable devices configured to alter the human control unit 200 between more than two modes. In some examples, a first actuation of the actuatable device 205 may switch the human control unit 200 to be operable in the first mode and upon a second actuation, in the second mode. In some examples, an actuation of a first device allows for the human control unit to be operable in the first mode and an actuation of a second device allows for the human control unit 200 to be operable in the second mode.

In some examples, the display 204 is configured to display at least one parameter of the apparatus. This may allow for a user of the human control unit 200 to see, and control, a wanted parameter. The parameter may be any suitable parameter such as, for example, distance, force, pressure and the like. The parameter may be displayed in discrete numbers, as a percentage of a possible maximum limit, as a moveable bar, as an element which increases in area as the parameter increases, or via any other suitable method.

In some examples, the transceiver is configured to send a control signal to the apparatus and/or receive a signal from the apparatus. This may allow for the human control unit 200 to control the apparatus or send a status indication to the apparatus and/or receive a controlling signal or receive a status indication from the apparatus. This may allow for the remote controlling of the apparatus by the human control unit 200.

In some examples, upon a rotation of the rotatable member 202, a parameter of the apparatus is altered. This may allow for the user to accurately and precisely control the parameter. In some examples, the rotatable member 202 comprises preset positions in which the member 202 can be. Additionally or alternatively, the member 202 may be rotatable continuously and comprise a, theoretically, infinite number of positions. That is to say, in the preset configuration, the rotatable member 202 “clicks” from one position to another, whereas in the continuous configuration, the movement of the rotatable member 202 is smooth.

In some examples, the rotatable member 202 is configured to provide haptic feedback to the user of the human control unit 200. This may allow for the user to have a realistic feel of the forces that are occurring at the apparatus. This haptic feedback is preferably proportional to a pressure value measured at the apparatus 100. In particular, this pressure may be the pressure measured by the first and/or second and/or third pressure sensors 104, 105 mentioned above. This may mean that for each increase in pressure, the resistive force felt by the user is increased. This may increase the accuracy of the procedure as the user can feel what is happening at the apparatus. The haptic feedback may be provided by the haptic feedback unit. The haptic feedback may be in the form of the user finding it easier/harder to rotate the member 202 based on the parameter and/or an increase/decrease in vibration of the member 202 as the parameter changes.

In some examples, as the parameter increases, the force of the haptic feedback provided to the user also increases. As a non-limiting example, should the parameter be 0 N, the user may not feel any haptic feedback. As the parameter increases to 5 N, the user may feel a haptic force of 2 N acting on the rotatable member 202, and as the parameter increases to 10 N, the force felt by the user may be 4 N. This may again help the user control the procedure, as at high parameters, it may be difficult to rotate the member 202. This, in turn, may improve the safety of the procedure, as the user may not be able to alter the parameter to dangerous levels.

In some examples, a change in the haptic feedback provided to the user is non-linear with respect to the change in the parameter. As a non-limiting example, should the parameter be 0 N, the user may not feel any haptic feedback. As the parameter increases to 5 N, the user may feel a haptic force of 2 N acting on the rotatable member 202, and as the parameter increases to 10 N, the force felt by the user may be 6 N. This may again help the user control the procedure, as at high parameters, it may be difficult to rotate the member. This, in turn, may improve the safety of the procedure as the user may not be able to alter the parameter to dangerous levels.

In some examples, as the parameter changes, the scalability of the rotation member 202 may alter. As an example, if the parameter relates to force, in order to go from 0 N to 5 N, a single full rotation of the rotatable member 202 may be needed. Between 5 N and 10 N, 2 full rotations of the rotatable member 202 may be needed, i.e. each rotation increases the force by 2.5 N. After 10 N, a single full rotation of the rotatable member 202 may be needed in order to increase the force by 1 N. In some examples, the scalability may be step-wise, or it may be a continuous curve. This, in turn, may improve the safety of the procedure, as the user may not be able to alter the parameter to dangerous levels.

In some examples, the human control unit 200 further comprises a timing module configured to be activated upon an actuation of a second actuatable device, and wherein an output of the timing module is configured to be displayed on the display 204. This may allow the user to control time-sensitive operations and see how long a current procedure has been going on for. In some examples, the timing module is activated upon actuation of an actuatable device, as mentioned above. Additionally or alternatively, the timing module may be activated upon the parameter reaching a predetermined value.

In some examples, the display 204 is further configured to display a graph with the output of the timing module on a first axis, and the at least one parameter of the apparatus on a second axis. This may allow the user to control time- and parameter-sensitive operations and see how long a current procedure has been going on for, and how long the presently controlled parameter has been at its current level. The graph may show only the last, for example, 60 seconds, or may show the entirety of the time the timing module has been activated.

In some examples, the human control unit 200 is configured to control a characteristic of the apparatus upon actuation of a third actuatable device. For example, the characteristic may relate to a movement of the apparatus, an activation of a feature, a deactivation of a feature, or any other suitable characteristic. This may allow for a user of the human control unit 200 to have greater control over the apparatus and more precisely control said apparatus.

In some examples, the rotatable member 202 is single use and/or disposable. This may allow for sterility to be maintained. In particular, the rotatable member may comprise any suitable plastic and/or metal.

In some examples, the rotatable member 202 comprises a circular cross-section or a cross shaped cross-section. This may help the moveable member 202 to be rotated quicker, if needed, and also help to move the moveable member 202 should the user be wearing gloves. Additionally, the alterability of the moveable member 202, and therefore the cross-section, may allow for users to switch moveable members 202 depending on personal preference. Although circular and cross cross-sections are mentioned, the skilled person understands that the cross-section can be any suitable cross-section. In some examples, the moveable member 202 may further comprise dimples to help rotation, and to orientate the user

In some examples, the human control unit 200 further comprises a light source, and wherein at least one characteristic of the light source changes based on the parameter of the apparatus. This light source may give a visual indication to the user as to the state of the parameter. For example, if the parameter is within normal limits, a green light may be indicated. As the parameter gets close to its upper allowable limit, the light may turn yellow, and as the parameter exceeds its limit, the light may turn red. This may allow for the user to refrain from exceeding the limits of a parameter. Additionally or alternatively, additional haptic feedback may be provided by vibrating the rotatable member, similar to a stick-shaker in an aircraft, and/or an audio indication may also be given. This may improve the safety of the procedure as the user is given warning of when limits are going to be exceeded/have been exceeded.

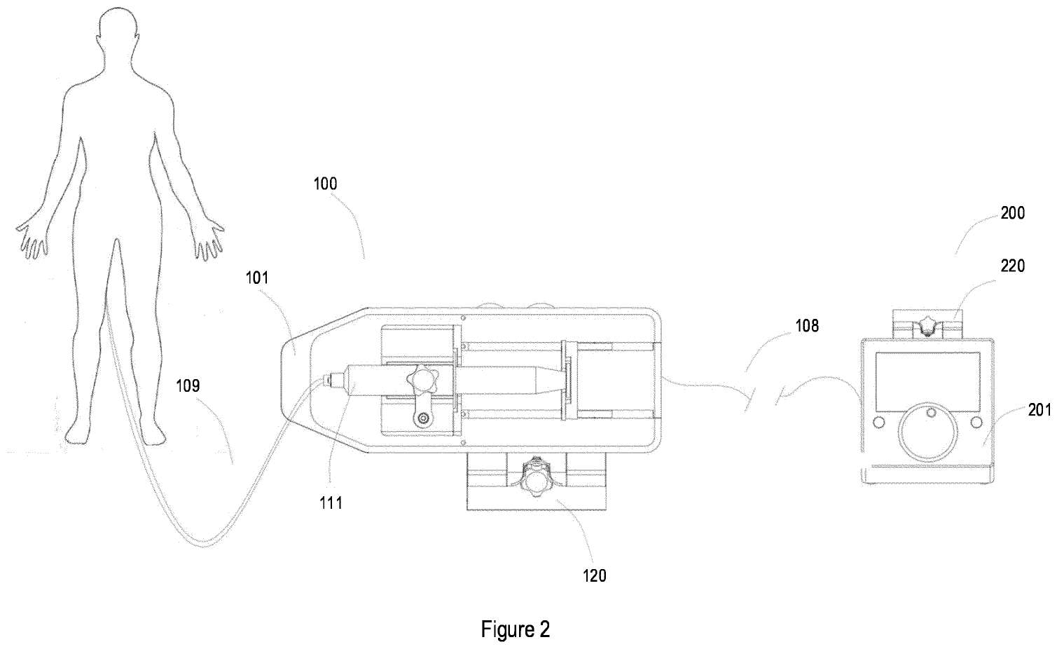

FIG. 2 shows a schematic view of an apparatus for endovascular procedures according to some example implementations as described herein.

The apparatus 100 and human control unit 200 of FIG. 2 are generally the same as those shown in FIG. 1. However, both the apparatus 100 and the human control unit comprise respective mounting rail adapters 120, 220. This may allow for both the apparatus 100 and the human control unit 200 to be moved according to the procedure. In some examples, the apparatus 100 may be mounted to a moveable rail, thereby allowing for the apparatus 100 to be moved during use. This may be particularly helpful during certain procedures. Likewise, the human control unit 200 may also be mounted on a similar moving rail. In some examples, the rail is static, and the apparatus 100 and/or the human control unit 200 need to be manually moved along the rail. A clamp style device is shown in FIG. 2. However, it is to be understood that any suitable method of clamping, and any suitable adapter shape, may be used.

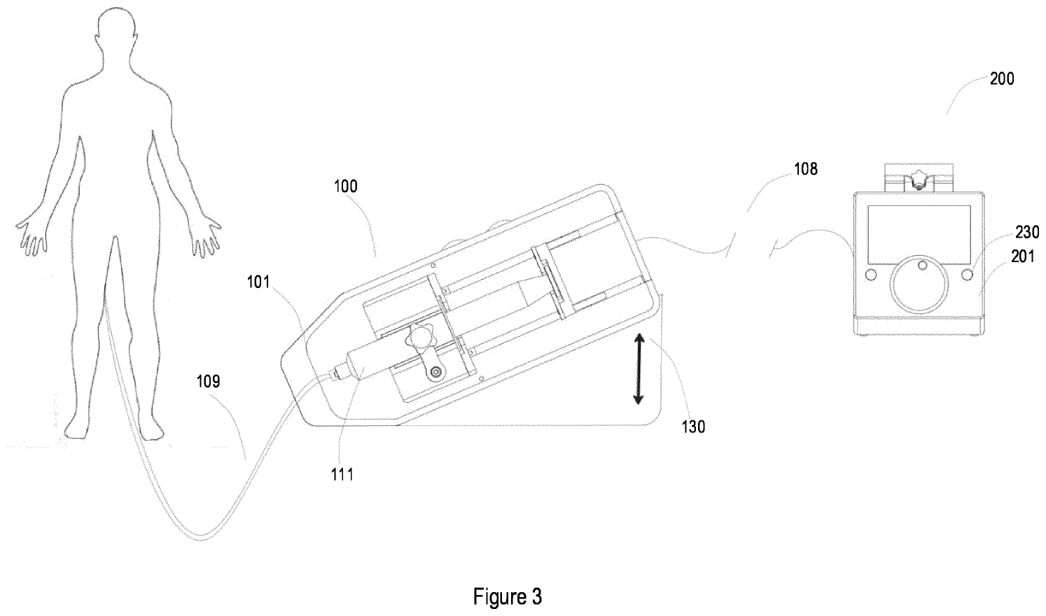

FIG. 3 shows a schematic view of an apparatus for endovascular procedures according to some example implementations as described herein.

In some examples, the moveable base 100 further comprises a rotational gear, and is configured to rotate the moveable base about a fixed point on the moveable base. The rotational gear may rotate the entire base 101, or, in some examples, a portion of the moveable base 101. Preferably, the portion of the moveable base 101 that is rotated comprises the connector 103 described above. The rotational gear may be any suitable known rotational gear that allows for continuous, non-stepped movement of the connector and/or the base. As the rotational gear is able to be moved in very small increments, the connector and/or the base is therefore moved in very small increments, thereby allowing for the syringe, and the apparatus, to be accurately and precisely used. The rotational gear allows at least the portion of the base to rotate in the direction of the arrow shown in FIG. 3. That is, the at least a portion of the base 101 is preferably configured to be rotated in a lateral axis of the syringe 111. In some examples, the rotational gear may, additionally or alternatively, be configured to rotate at least a portion of the base 101 in a longitudinal axis and/or a vertical axis of the syringe 111. In some examples, there are a plurality or rotational gears.

In some examples, the rotational movement of the moveable base 101 is further configured to rotate the moveable base about a proximal end of the syringe 111. That is to say, the end of the syringe 111 coupled to the elongated medical member 109 is substantially kept in the same place during the rotation of the moveable base 101. This may therefore allow for the relative position of the elongated medical member 109 with respect to both the syringe 111, a surface on which the apparatus is placed, and the patient to remain substantially stable. This may be important during procedures, as movement of the elongated medical member 109 within the patient may cause unwanted effects.

In some examples, the moveable base 101 is rotated, via the rotational gear, when an instruction to rotate the moveable base is executed by the control unit. This may allow for the rotational gear to be actuated. Connections between gears and control units, and methods of causing gears to undergo instructions executed by the control unit are known to the skilled person.

In some examples, the movable base 101 is repeatedly rotated, via the rotational gear, from a fifth position to a sixth position, and back to a fifth position, when an instruction to repeatedly rotate the moveable base is executed by the control unit. That is to say, upon execution of a single instruction, the moveable base 101 may be repeatedly rotated through a predetermined angle. This may allow for any air bubbles within the syringe 111 and/or the elongated medical member 109 to be moved to within the syringe 111. This may be important as air bubbles within the elongated medical member 109 and/or syringe 111 may cause unwanted effects to the patient during the procedure. Resultantly, the procedure may be safer.

FIG. 4 shows a block diagram of an apparatus for endovascular procedures according to some example implementations as described herein.

As mentioned above, the apparatus 100 comprises a linear gear 102, a rotational gear 306, a transceiver 300, a control unit 302, a safety sensor 106, first and second pressure sensors 105, 310 and a third pressure sensor 104, in addition to the other features described herein. Communication between the various elements of the apparatus 100 is shown via the arrows in FIG. 4. Additionally, the communication line 108 is shown. The transceiver 300 allows for instructions and/or control signals and/or signals to be sent and/or received from an external source, such as, for example, the human control unit 200.

FIG. 5 shows a block diagram of a human control unit 200 according to some example implementations as described herein.

As mentioned above, the human control unit 200 comprises a transceiver 400, a control unit 402, a haptic feedback unit 404, a light source 406 and a timing module 408, in addition to the other features described herein. Communication between the various elements of the apparatus 100 is shown via the arrows in FIG. 5. Additionally, the communication line 108 is shown. The transceiver 400 allows for instructions and/or control signals and/or signals to be sent and/or received from an external source, such as, for example, the apparatus 100.

The following examples are also encompassed by the present disclosure and may fully or partly be incorporated into embodiments.

1. An apparatus for an endovascular procedure, wherein the apparatus comprises:
a moveable base;
a drive unit coupled to at least a first portion of the moveable base and configured to move the first portion of the moveable base;
a syringe comprising a plunger, wherein the syringe is couplable to the base;
and
a first control unit configured to control a movement of at least a first portion of the movable base;
wherein, upon an instruction provided by the first control unit to the drive unit, at least the first portion of the moveable base is configured, based on a movement of the first portion of the moveable base, to move the plunger from a first position to a second position and/or the second position to the first position, and wherein the first and second positions are different positions.
   2. The apparatus of clause 1, wherein the apparatus further comprises an elongated medical member couplable to a proximal end of the syringe
   3. The apparatus of clause 1 or 2, wherein the moveable base further comprises a connector configured to couple at least the plunger of the syringe to the first portion of the moveable base.
   4. The apparatus of clause 3, wherein the drive unit comprises a linear gear coupled to the connector, and wherein the linear gear is configured to move the connector from a third position to a fourth position and/or the fourth position to the third position, upon the instruction being provided by the first control unit to the drive unit, wherein, when the connector is in the third position, the plunger is in the first position, and wherein, when the connector is in the fourth position, the plunger is in the second position.
   5. The apparatus of clause 4, wherein the connector is configured to be moved from the third position to the fourth position and/or the fourth position to the third position, via the linear gear, when the instruction to move the connector from the third position to the fourth position and/or the fourth position to the third position is executed by the first control unit.
   6. The apparatus of any one of the preceding clauses, wherein the first control unit comprises a processor and a memory, wherein the memory is configured to store the instruction and the processor is configured to execute the instruction stored in the memory and/or an instruction received from an external source.
   7. The apparatus of any one of the preceding clauses, wherein the drive unit comprises a rotational gear, and wherein the rotational gear is configured to rotate at least a second portion of the moveable base about a fixed point on and/or a fixed axis through the moveable base.
   8. The apparatus of clause 7, wherein the rotational movement of the second portion of the moveable base is further configured to rotate the moveable base about a proximal end of the syringe.
   9. The apparatus of clause 7 or 8, wherein the second portion of the moveable base is configured to be rotated, via the rotational gear, when an instruction to rotate at least the second portion of the moveable base is executed by the first control unit.
   10. The apparatus of any one of clauses 7 to 9, wherein at least the second portion of the movable base is configured to be repeatedly rotated, via the rotational gear, from a fifth position to a sixth position, and back to the fifth position, when an instruction to repeatedly rotate at least the second portion of the moveable base is executed by the first control unit.
   11. The apparatus of any one of the preceding clauses, when dependent on clause 4 and/or clause 7, wherein the moveable base further comprises an actuatable device, wherein, upon actuation of the actuatable device, the first control unit is configured to execute an instruction to move the connector from the third position to the fourth position and/or the fourth position to the third position and/or rotate at least the second portion of the moveable base.
   12. The apparatus of any one of the preceding clauses, further comprising a safety sensor couplable to the first control unit, and wherein the safety sensor is configured to determine if the syringe is coupled to the moveable base.
   13. The apparatus of any one of the preceding clauses, further comprising a first pressure sensor communicatively couplable to the first control unit, wherein the first pressure sensor is couplable to the syringe, and wherein the first pressure sensor is configured to measure a pressure within the syringe based on a movement of the plunger of the syringe, wherein the first pressure sensor is configured to measure pressure difference between the pressure within the syringe and a predetermined pressure value.
   14. The apparatus of clause 13, when dependent on clause 4, wherein the first pressure sensor is configured to measure the pressure based on a movement of the connector from the third position to the fourth position and/or the fourth position to the third position.
   15. The apparatus of any one of the preceding clauses, wherein the syringe is at least partially filled with a liquid.
   16. The apparatus of clause 15, wherein the liquid comprises microspheres.
   17. The apparatus of any one of the preceding clauses, when dependent on clause 2, and when dependent on clause 15 or 16, further comprising a second pressure sensor communicatively couplable to the first control unit, wherein the second pressure sensor is couplable to the elongated medical member, wherein the elongated medical member is at least partially filled with the liquid, and wherein the second pressure sensor is configured to measure a pressure of the liquid within the elongated medical member.
   18. The apparatus of any one of the preceding clauses, further comprising a force sensor configured to measure a force exerted by the plunger on the syringe.
   19. The apparatus of any one of the preceding clauses, wherein a distal end of the syringe is configured to be elevated above the proximal end of the syringe during use of the apparatus, the distal end is an end of the syringe comprising the plunger, wherein the distal and proximal ends are at two different end portions of the syringe.
   20. The apparatus of any one of the preceding clauses, further comprising an expandable medical member couplable to the elongated medical member, wherein a first end of the elongated medical member is couplable to the proximal end of the syringe, and the expandable medical member is couplable to a second end of the elongated medical member, wherein the first and second ends are at opposite ends of the elongated medical member.
   21. The apparatus of clause 20, wherein a diameter of the expandable medical member is dependent on a pressure of a liquid within the elongated medical member.
   22. The apparatus of any one of the preceding clauses, wherein the apparatus is additionally or alternatively suitable for angioplasty.
   23. The apparatus of any one of the preceding clauses, wherein the apparatus is additionally or alternatively suitable for embolization.
   24. A system for an endovascular procedure comprising:
the apparatus of any one of clauses 1 to 23; and
a human control unit comprising a second control unit;
wherein the human control unit is located at a first location and the apparatus is located at a second location; and
wherein the first and second locations are different locations.
   25. A human control unit for manipulating an apparatus for an endovascular procedure, the human control unit comprising:
a housing;
a control unit comprising a transceiver;
a moveable member couplable to the housing and moveable by a user of the human control unit; and
a haptic feedback unit communicatively couplable to the control unit and couplable to the moveable member;
wherein, upon a movement of the moveable member, the transceiver is configured to provide a signal to the apparatus for altering a parameter of the apparatus, and wherein the haptic feedback unit is configured to provide haptic feedback via the moveable member to the user of the human control unit.
   26. The human control unit of claim 1, wherein the haptic feedback provided to the user changes based on the altered parameter.
   27. The human control unit of clause 25 or 26, wherein the moveable member comprises a rotatable member, and wherein the movement of the moveable member comprises a rotation of the rotatable member.
   28. The human control unit of any one of clauses clause 25 to 27, further comprising a first actuatable device, wherein upon actuation of the actuatable device, the human control unit changes from a first mode to a second mode.
   29. The human control unit of any one of clauses 25 to 28, wherein the human control unit is couplable to the apparatus.
   30. The human control unit of clause 29, wherein the transceiver is further configured to receive an apparatus signal from the apparatus.
   31. The human control unit of any one of clauses 25 to 30, wherein, upon the parameter increasing, the haptic feedback unit is configured to increase a force applied via the moveable member to the user.
   32. The human control unit of any one of clause 25 to 31, wherein a change in the haptic feedback provided to the user is non-linear with respect to a change of a value of the parameter.
   33. The human control unit of any one of clauses 25 to 32, further comprising a display couplable to the control unit and the housing.
   34. The human control unit of clause 33, wherein the display is configured to display the parameter or a value associated with the parameter of the apparatus.
   35. The human control unit of clause 33 or 34, wherein the human control unit further comprises a timing module configured to be activated upon an actuation of a second actuatable device, and wherein the display is configured to display an output of the timing module.
   36. The human control unit of clause 35, wherein the display is further configured to display a graph with the output of the timing module on a first axis, and the parameter of the apparatus on a second axis.
   37. The human control unit of any one of clauses 25 to 36, when dependent on clause 29, wherein the human control unit is configured to control a characteristic of the apparatus upon actuation of a third actuatable device.
   38. The human control unit of any one of clauses 25 to 37, wherein the moveable member is single use and/or disposable.
   39. The human control unit of any one of clause 25 to 38, wherein the moveable member comprises a circular cross-section or a cross-shaped cross-section.
   40. The human control unit of any one of clause 25 to 39, wherein the human control unit further comprises a light source, and wherein at least one characteristic of the light source changes based on a change of a value of the parameter of the apparatus.
   41. The human control unit of any one of clause 25 to 40, wherein the moveable member comprises a plurality of predefined positions, wherein the moveable member is clickable between said plurality of predefined positions and/or wherein the moveable member is continuously moveable.
   42. A system for manipulating an apparatus for an endovascular procedure, the system comprising:
an apparatus; and
the human control unit of any one of clauses 25 to 41;
wherein the human control unit is located at a first location and the apparatus is located at a second location; and
wherein the first and second locations are different locations.
   43. The system of clause 42, wherein the apparatus and the human control unit are communicatively couplable to one another via wireless means.
   44. The system of clause 42 or 43, wherein the apparatus comprises a second transceiver, wherein the parameter alterable by the movement of the moveable member is a pressure, and wherein upon the pressure falling below a predetermined pressure, the apparatus is configured to send a signal to the human control unit for preventing the human control unit from manipulating the apparatus.

1. A human control unit for manipulating an apparatus for an endovascular procedure, the human control unit comprising:
a housing;
a control unit comprising a transceiver;
a moveable member couplable to the housing and moveable by a user of the human control unit; and
a haptic feedback unit communicatively couplable to the control unit and couplable to the moveable member;
wherein, upon a movement of the moveable member, the transceiver is configured to provide a signal to the apparatus for altering a parameter of the apparatus, and wherein the haptic feedback unit is configured to provide haptic feedback via the moveable member to the user of the human control unit.

2. The human control unit of claim 1, wherein the haptic feedback provided to the user changes based on the altered parameter.

3. The human control unit of claim 1, wherein the moveable member comprises a rotatable member, and wherein the movement of the moveable member comprises a rotation of the rotatable member.

4. The human control unit of claim 1, further comprising a first actuatable device, wherein upon actuation of the actuatable device, the human control unit changes from a first mode to a second mode.

5. The human control unit of claim 1, wherein the human control unit is couplable to the apparatus.

6. The human control unit of claim 5, wherein the transceiver is further configured to receive an apparatus signal from the apparatus.

7. The human control unit of claim 1, wherein, upon the parameter increasing, the haptic feedback unit is configured to increase a force applied via the moveable member to the user.

8. The human control unit of claim 1, wherein a change in the haptic feedback provided to the user is non-linear with respect to a change of a value of the parameter.

9. The human control unit of claim 1, further comprising a display couplable to the control unit and the housing.

10. The human control unit of claim 9, wherein the display is configured to display the parameter or a value associated with the parameter of the apparatus.

11. The human control unit of claim 9, wherein the human control unit further comprises a timing module configured to be activated upon an actuation of a second actuatable device, and wherein the display is configured to display an output of the timing module.

12. The human control unit of claim 11, wherein the display is further configured to display a graph with the output of the timing module on a first axis, and the parameter of the apparatus on a second axis.

13. The human control unit of claim 1, wherein the human control unit is couplable to the apparatus, and wherein the human control unit is configured to control a characteristic of the apparatus upon actuation of a third actuatable device.

14. The human control unit of claim 1, wherein the moveable member is single use and/or disposable.

15. The human control unit of claim 1, wherein the moveable member comprises a circular cross-section or a cross-shaped cross-section.

16. The human control unit of claim 1, wherein the human control unit further comprises a light source, and wherein at least one characteristic of the light source, in particular a brightness and/or color of light radiated via the light source, changes based on a change of a value of the parameter of the apparatus.

17. The human control unit of claim 1, wherein the moveable member comprises a plurality of predefined positions, wherein the moveable member is clickable between said plurality of predefined positions and/or wherein the moveable member is continuously moveable.

18. A system for manipulating an apparatus for an endovascular procedure, the system comprising:
an apparatus; and
a human control unit comprising:
a housing;
a control unit comprising a transceiver;
a moveable member couplable to the housing and moveable by a user of the human control unit; and
a haptic feedback unit communicatively couplable to the control unit and couplable to the moveable member;
wherein, upon a movement of the moveable member, the transceiver is configured to provide a signal to the apparatus for altering a parameter of the apparatus, and wherein the haptic feedback unit is configured to provide haptic feedback via the moveable member to the user of the human control unit;
wherein the human control unit is located at a first location and the apparatus is located at a second location; and
wherein the first and second locations are different locations.

19. The system of claim 18, wherein the apparatus and the human control unit are communicatively coupled to one another.

20. The system of claim 18, wherein the apparatus comprises a second transceiver, wherein the parameter alterable by the movement of the moveable member is a pressure, and wherein upon the pressure falling below a predetermined pressure, the apparatus is configured to send a signal to the human control unit for preventing the human control unit from manipulating the apparatus.
